SEQUENCE LISTING (1 sequence)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2011/0171168 A1
(43) Pub. Date: Jul. 14, 2011

(54) HUMAN G-CSF ANALOGS AND METHODS OF MAKING AND USING THEREOF

(75) Inventor: Darryl L. Carter, Owings Mills, MD (US)

(73) Assignee: NORA THERAPEUTICS, INC., Owings Mills, MD (US)

(21) Appl. No.: 12/682,008

(22) PCT Filed: Jul. 21, 2008
(86) PCT No.: PCT/US08/70668

§ 371 Date: May 19, 2010

Related U.S. Application Data
(63) Continuation-in-part of application No. 12/120,022, filed on May 13, 2008, now abandoned.

(51) Int. Cl. A61K 38/19; A61P 37/06; A61P 15/06

(52) U.S. Cl. 424/85.1

(57) ABSTRACT

An analog of human granulocyte colony stimulating factor (hG-CSF analog) is disclosed. The hG-CSF analog comprises an amino acid sequence that differs from the wild-type hG-CSF sequence at position 17 and at least one other position, and is capable of preventing trophoblast cell apoptosis. Also disclosed is pharmaceutical compositions comprising the hG-CSF analog, polynucleotides encoding the hG-CSF analog, expression vectors containing the polynucleotides, host cells containing the expression vectors, as well as a method for preventing spontaneous abortion using the hG-CSF analog.

### FIELD

The present invention relates to compositions capable of preventing trophoblast apoptosis; particularly, the compositions can be used for preventing spontaneous abortion, complications associated with threatened spontaneous abortion, and implantation failure and miscarriage during assisted reproduction.

### BACKGROUND

Spontaneous abortion occurs in 15% of diagnosed pregnancies in women between fifteen and forty-five years of age (Griebel C P, et al., Am Fam Physician. 2005 Oct. 1; 72(7):1243-5, Review). Recurrent spontaneous abortions are defined as the spontaneous loss of three or more pregnancies and occur in about 1-5% of these women. The risk of pregnancy loss roughly doubles after one spontaneous abortion (Stephenson M, Kutteh, Clin Obstet Gynecol. 2007 March; 50(1):132-45. Review).

Although many pregnancies lost in the first trimester are due to fetal chromosomal abnormalities, spontaneous abortion, the loss of the product of conception prior to the 20th week of pregnancy, is often a disorder of unknown etiology. It has been theorized that spontaneous abortions are a natural rejection of a fetus with abnormalities incompatible with life; however, this theory has yet to be substantiated. (Sullivan A E, et al., Obstet. Gynecol. 2004 October; 104(4):784-8).

Risk factors for abortion include age, weight and overall health of the woman. The prevalence of spontaneous abortion increases with increasing maternal age, although not with gravidity. The risk begins to increase rapidly at age 35 years. The risk of euploid spontaneous abortion at age 40 is approximately twice that at age 20. As families are planned later and later in life, the frequency of spontaneous abortion will only increase without effective methods of prevention.

Threatened abortion generally presents as cramping and bleeding for which treatment is bed rest. This conservative treatment provides palliative care for the mother but does little to alter the outcome. The use of hormones is generally contraindicated due to the risk of congenital anomalies, including malformation of the vessels of the heart of the embryo and possible genital abnormalities in female offspring.

The loss of a desired pregnancy takes a tremendous emotional toll on hopeful and expectant parents. Loss of a pregnancy can lead to feelings of inadequacy, hopelessness and guilt, which can have a devastating effect on individuals and on a marriage.

New methods and compositions are always needed to reduce risks associated with pregnancy to the health of the mother and fetus. Effective prevention of spontaneous abortion can allow women, especially women at risk, to have successful pregnancies.

### SUMMARY

One aspect of the present invention relates to an analog of human granulocyte colony stimulating factor (hG-CSF analog) comprising an amino acid sequence that differs from the sequence in SEQ ID NO: 1 at position 17 and at least one other position, wherein said hG-CSF analog is capable of preventing trophoblast cell apoptosis.

Another aspect of the present invention relates to a pharmaceutical composition comprising the hG-CSF analog polypeptide described above and a pharmaceutically acceptable carrier.

Another aspect of the present invention relates to a kit comprising one or more unit dosages of the pharmaceutical composition which comprises the hG-CSF analog polypeptide described above and a pharmaceutically acceptable carrier.

Another aspect of the present invention relates to a polynucleotide encoding the hG-CSF analog described above.

Another aspect of the present invention relates to an expression construct containing the polynucleotide described above.

Another aspect of the present invention relates to a host cell containing the polynucleotide described above.

Yet another aspect of the present invention relates to a method for preventing spontaneous abortion, complications associated with threatened spontaneous abortion, and implantation failure and miscarriage during assisted reproduction using the hG-CSF analog of the present invention.

### DETAILED DESCRIPTION

The practice of the present invention will employ, unless otherwise indicated, conventional methods of molecular biology, cell biology, immunology, obstetrics and gynecology, and within the skill of the art. Such techniques are explained fully in the literature. All publications, patents and patent applications cited herein, whether supra or infra, are hereby incorporated herein by reference in their entirety.

As used herein, the following terms shall have the following meanings:

The term “nucleotide sequence” is intended to indicate a consecutive stretch of two or more nucleotide molecules. The nucleotide sequence may be of genomic, cDNA, RNA, semi-synthetic or synthetic origin, or any combination thereof.

“Cell,” “host cell,” “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “conjugate” is intended to indicate a heterogeneous molecule formed by the covalent attachment of one or more polypeptides, typically a single polypeptide, to one or more non-polypeptide moieties such as polymer molecules, lipophilic compounds, carbohydrate moieties or organic derivatizing agents. The term “covalent attachment” means that the polypeptide and the non-polypeptide moiety are either directly covalently joined to one another, or else are indirectly covalently joined to one another through an intervening moiety or moieties, such as a bridge, spacer, or linkage moiety or moieties. Preferably, the conjugate is soluble at relevant concentrations and conditions, i.e., soluble in physiological fluids such as blood. The term “non-conjugated polypeptide” may be used about the polypeptide part of the conjugate.

The term “recombinant protein” refers to a protein made using recombinant techniques, i.e., through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5%, by weight of the total protein in a given sample. A substantially pure protein comprises at least about 75% by weight of the total protein, with at least about 80% being preferred, and at least about 90% being particularly preferred.

The term “treat,” “treating” or “treatment,” as used herein, refers to a method of alleviating or abrogating a disorder and/or its attendant symptoms. The terms “prevent,” “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “spontaneous abortion” refers to delivery or loss of the product of conception before the 20^{th} week of pregnancy. The term “spontaneous abortion” includes but is not limited to miscarriage, threatened abortion, inevitable spontaneous abortion, incomplete spontaneous abortion, habitual or recurrent spontaneous abortion or missed abortion.

The term “habitual spontaneous abortion” or “recurrent spontaneous abortion” refers to three or more consecutive spontaneous abortions.

The term “complications associated with threatened abortion” refers to well-known obstetrical complications that can result from threatened abortion and which pose a significant risk of morbidity or mortality to the fetus and/or the mother. The term “complications associated with threatened abortion” includes but is not limited to placenta previa, placental abruption, preeclampsia and preterm labor.

The term “in vitro fertilization” refers to the procedure involving ovarian hyperstimulation, oocyte retrieval from the mother-to-be or a donor, fertilization outside the subject's body, embryo culture and embryo transfer. As used herein, embryo transfer refers to the procedure involving transfer to a subject's uterus of the developing or cleaving embryos or pre-embryos, also termed “preimplantation embryos.”

The term “implantation failure” refers to the failure of an embryo produced by assisted reproduction to implant normally or at all in the uterus of a recipient subject.

The term “miscarriage in assisted reproduction” refers to the delivery or loss of the transferred embryo before the 20^{th} week of pregnancy.

The term “frozen embryo transfer” refers to a procedure where cryopreserved pre-implantation embryos that are produced outside of a subject's body are transferred to a subject's uterus.

The term “ICSI” refers to a procedure (intracytoplasmic sperm injection), which involves mechanical injection of sperm into the oocyte.

The term “IUI” refers to procedure in which a fine catheter (tube) is inserted through the cervix (the natural opening of the uterus) into the uterus (the womb) to deposit a sperm sample directly into the uterus.

The term “artificial insemination” refers to a fertilization procedure in which sperm is artificially placed into a woman's cervix or uterus.

The term “ZIFT” refers to a procedure in which the zygote, in its pronuclear stage of development, is transferred into the Fallopian tube.

The term “GIFT” refers to a procedure in which the male gamete (i.e., sperm), is transferred into the Fallopian tube.

The term “assisted reproduction” refers to clinical and laboratory techniques used to enhance fertility in humans and animals, including, but not limited to, in vitro fertilization, frozen embryo transfer, ICSI, GIFT, ZIFT, IUI, artificial insemination, hormone-induced superovulation, and the like.

The term “hormone-induced superovulation” refers to ovulation of a super normal number of ova; usually the result of administration of exogenous gonadotropins.

The term “human granulocyte-colony stimulating factor” or “hG-CSF” refers to the polypeptide having the amino acid sequence of SEQ ID NO:1.

The term “hG-CSF analog” refers to a polypeptide having an amino acid sequence that differs from the amino acid sequence of the wild-type hG-CSF at one or more locations while exhibiting G-CSF activity.

The term “exhibiting G-CSF activity” refers to the polypeptide or conjugate having one or more of the functions of native G-CSF, in particular hG-CSF with the amino acid sequence shown in SEQ ID NO:1, including the capability to bind to a G-CSF receptor (Fukunaga, et al., J. Bio. Chem., 265:14008, 1990). The G-CSF activity is conveniently assayed using the primary assay described in the Materials and Methods section hereinafter. The polypeptide “exhibiting” G-CSF activity is considered to have such activity when it displays a measurable function, e.g., a measurable proliferative activity or a receptor binding activity (e.g., as determined by the primary assay described in the Materials and Methods section). The polypeptide exhibiting G-CSF activity may also be termed “G-CSF” or “G-CSF molecule” herein.

The term “granulocyte” refers to a blood cell containing granules, especially a leukocyte (white blood cell or corpuscle) containing neutrophil, basophil or eosinophil granules in its cytoplasm.

The term “effective amount” refers to that amount of an active agent being administered sufficient to reduce the risk or prevent development of the disorder being treated.

The term “subject” refers to animals such as mammals, including, but not limited to, primates (such as humans), cows, sheep, goats, horses, dogs, cats, rabbits, guinea pigs, rats, mice and the like. In preferred embodiments, the subject is a human female.

The term “label” refers to a display of written, printed or graphic matter on the immediate container of an article, for example, the written material displayed on a vial containing a pharmaceutically active agent.

The term “labeling” refers to all labels and other written, printed or graphic matter on any article or any of its containers or wrappers or accompanying such article, for example, a package insert or instructional videotapes or computer data storage devices, such as CDs and DVDs, accompanying or associated with a container of a pharmaceutically active agent.

While not intending to be bound by any particular theory of operation, as discussed above, it is believed that spontaneous abortion is caused by or associated with an inappropriate Th1 immune response. It is believed that administration of G-CSF can prevent spontaneous abortion by reducing the inappropriate Th1 immune response and/or increasing a Th2 immune response in a subject at risk for spontaneous abortion. It has been observed that G-CSF can mobilize peripheral blood stem cells, and that these stem cells, when administered to a subject, can shift the subject's immune response toward a Th2 response. Therefore, it is also possible to prevent spontaneous abortion by administration of G-CSF mobilized peripheral blood stem cells. In addition, histophatologic examination of the products of conception from spontaneous pregnancy losses reveals that trophoblast cell apoptosis is a prominent feature, the trophoblast representing the microanatomic maternal fetal interface. The present invention seeks to prevent spontaneous pregnancy loss by preventing trophbolast apoptosis with an hG-CSF analog.

G-CSF is pleiotropic cytokine. Since its initial description as a hematopoetic growth factor that selectively stimulates neutrophil proliferation, maturation and survival, numerous other effects of G-CSF have been discovered in non hematopoietic cells, tissues, and organs. The G-CSF receptor is widely distributed in various tissues and organs in mammals. At least seven isoforms of the G-CSF receptor have been identified. Most of these isoforms have identical extracellular and transmembrane domains and differ only in their cytoplasmic tails, the portion of the receptor directly responsible for intracellular signaling. Trophblastic cells express an isoform of the hG-CSF receptor that represents a different isoform from that found in neutrophils.

One aspect of the present invention is directed to an hG-CSF analog comprising an amino acid sequence that differs from the sequence in SEQ ID NO:1 at position 17 and at least another position, wherein said analog is capable of inhibiting trophoblast cell apoptosis.

In one embodiment, the hG-CSF analog comprises a polypeptide sequence that differs from the sequence in SEQ ID NO:1 at positions 17 and 38, and at least another position.

In another embodiment, the hG-CSF analog comprises a polypeptide sequence that differs from the sequence in SEQ ID NO:1 at positions 17, 38 and 58.

In another embodiment, the hG-CSF analog comprises a polypeptide sequence that differs from the sequence in SEQ ID NO:1 at positions 17, 38 and 53.

In another embodiment, the hG-CSF analog contains, at position 17, an amino acid selected from the group consisting of leucine, methionine, glutamine, tryptophane, alanine, tyrosine, serine, lysine, glutamine, threonine, asparagine, and histidine.

In another embodiment, the hG-CSF analog contains a substitution at position 38.

In another embodiment, the hG-CSF analog contains a substitution at position 53.

In another embodiment, the hG-CSF analog contains a substitution at position 58.

In another embodiment, the hG-CSF analog of the present invention contains substitutions that are made in amino acids that are on the surface of the protein and that are not involved in intramolecular hydrogen bonding. Preferred sites include positions 12, 16, 18, 23, 32, 33, 43, 44, 45, 46, 52, 57, 58, 71, 83, 90, 98, 101, 104, 108, 123, 137 and 159.

In another embodiment, the hG-CSF analog of the present invention contains substitutions that are made in amino acids that are on the surface of the protein and that are involved in intramolecular hydrogen bonding. Preferred sites include positions 22, 38, 39, 53, 77, 80, 93, 105, 115, 118, 122, 145 and 169.

The hG-CSF analog does not contain mutations that are known to disrupt the 3-dimensional conformation of G-CSF in a manner that impairs or reduces the affinity of G-CSF to its receptor, that impairs the ability of the G-CSF/G-CSF receptor complex to dimerize, or that significantly reduces the hG-CSF analog's stability (Reidhaar-Olson J F, et al., Biochemistry. 1996 Jul. 16; 35(28):9034-41). These excluded mutations will likely include mutations at the following 15 positions of SEQ ID NO:1, 15, 19, 25, 31, 34, 40, 47, 48, 49, 54, 112, 124, 142, 144 and 146.

A person of ordinary skill in the art would understand that modifications and changes can be made in the structure of the hG-CSF analog of the present invention and still obtain a molecule having desired biological activity (i.e., ability to inhibit trophoblast cell apoptosis). Because it is the interactive capacity and nature of a polypeptide that defines that polypeptide's biological activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art. It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is well-known in the art that an amino acid can be substituted by another amino acid having a similar hydropathic index and still obtain a functionally equivalent polypeptide. In such changes, the substitution of amino acids whose hydropathic indices are within +/−2 is preferred, those that are within +/−1 are particularly preferred, and those within +/−0.5 are even more particularly preferred. Substitution of like amino acids can also be made on the basis of hydrophilicity, particularly where the biological functional equivalent polypeptide, or polypeptide fragment, is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e., with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below).

TABLE 1
Amino Acid Substitutions

| Original Residue | Exemplary Residue Substitution |
|---|---|
| Ala | Gly; Ser |
| Arg | Lys |
| Asn | Gln; His |
| Asp | Glu |
| Cys | Ser; Ala |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser; Ala |
| Trp | Tyr |
| Tyr | Trp; Phe |
| Val | Ile; Leu |

The hG-CSF analog of the present invention may contain non-conservative changes. In a preferred embodiment, variant polypeptides differ from a native sequence by substitution, deletion or addition of five amino acids or fewer. The hG-CSF analog may also (or alternatively) be modified by, for example, the deletion or addition of amino acids that have minimal influence on the immunogenicity, secondary structure, tertiary structure, and hydropathic nature of the polypeptide.

The hG-CSF analog also includes a polypeptide that is modified from the original polypeptide by either natural process, such as post-translational processing, or by chemical modification techniques which are well known in the art. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a fluorophore or a chromophore, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

In one embodiment, the hG-CSF analog of the present invention is generated using an expression vector containing a polynucleotide sequence encoding the hG-CSF analog. The polynucleotide sequence encoding the hG-CSF analog is generated by introducing mutations into the coding sequence of a wild-type hG-CSF with standard techniques, such as site-directed mutagenesis and PCR-mediated mutagenesis. Alternatively, mutations can be introduced randomly along all or part of the coding sequence of the wild-type hG-CSF, such as by saturation mutagenesis, and the resultant mutants can be screened for biological activity to identify mutants that retain activity. Following mutagenesis, the hG-CSF analog can be expressed recombinantly and the activity of the protein can be determined.

In one embodiment, oligonucleotide primers are designed to introduce one or more amino acid mutations at the desired codon(s) of the coding sequence of the wild-type hG-CSF, which is cloned into an expression vector. Mutations will be confirmed by dideoxy DNA sequencing. Once DNA sequences have been confirmed, cells will be transfected with the expression vector. The expressed hG-CSF analog will be purified under conditions to minimize endotoxin contamination. A test for endotoxin will be performed by the Limulus amebocyte test. The hG-CSF analog will be tested for the ability to prevent apoptosis on JEG-3 cells exposed to recombinant human gamma interferon in in vitro culture. The detailed method will closely follow that of Sun, et al. (Sun Q H, et al., J Interferon Cytokine Res. 2007 July; 27(7):567-78). Briefly, coriocarinoma cells (JEG or JAR-3 cell lines) will be exposed to recombinant human gamma interferon in vitro at a concentration that has been shown to induce apoptosis of cytotrophoblast cells (100 IU per ml) for 72 hours. The JEG or JAR-3 cells will be maintained in a chemically defined serum-free culture media and will be grown in Teflon 24-well plates to prevent them from adhering. After 72 hours, the cell suspensions will be harvested and washed three times in PBS. Cells will then be stained with Annexin V and 7-AAD for analysis of cell death by flow cytometry (Lecoeur H, et al., J. Immunol. Methods. 1997 Dec. 1; 209(2):111-23). Cells that are Annexin V positive and 7-AAD negative will be scored as apoptotic. Cells that are negative for both Annexin V and 7-AAD will be scored as viable. Cells that are positive for both Annexin V and 7-AAD will be scored as nectrotic. The relative activity (the ratio of viable to apoptotic cells) of the analogs at various concentrations will be compared to that of gamma interferon alone and to a pseudowildtype hG-CSF analog. The pseudowildtype hG-CSF analog will contain a single substitution of an alanine for the native cysteine at position 17.

Alternative to recombinant expression, the hG-CSF analog can be synthesized chemically using standard peptide synthesis techniques.

The hG-CSF analog of the present invention also includes fusion proteins. A fusion hG-CSF analog typically contains an hG-CSF analog-related polypeptide and a non-hG-CSF analog-related polypeptide. The hG-CSF analog-related polypeptide may correspond to all or a portion of an hG-CSF analog. In a preferred embodiment, the fusion hG-CSF analog comprises at least one biologically active portion of an hG-CSF analog. Within the fusion protein, the term “operatively linked” is intended to indicate that the hG-CSF analog-related polypeptide and the non-hG-CSF analog-related polypeptide are fused in-frame to each other. The non-hG-CSF analog-related polypeptide can be fused to the N-terminus or C-terminus of the hG-CSF analog-related polypeptide.

A peptide linker sequence may be employed to separate the hG-CSF analog-related from non-hG-CSF analog-related components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the hG-CSF analog-related peptide and non-hG-CSF analog-related polypeptide; and (3) the lack of hydrophobic or charged residues that might react with the polypeptide functional epitopes. Preferred peptide linker sequences contain gly, asn and ser residues. Other near neutral amino acids, such as thr and ala may also be used in the linker sequence. Amino acid sequences which may be used as linkers are well known in the art. The linker sequence may generally be from 1 to about 50 amino acids in length. Linker sequences are not required when the hG-CSF analog-related polypeptide and non-hG-CSF analog-related polypeptide have non-essential N-terminal amino acid regions that can be used to separate the functional domains and prevent steric interference.

For example, in one embodiment, the fusion protein is a glutathione S-transferase (GST)-hG-CSF analog fusion protein in which the hG-CSF analog sequences are fused to the C-terminus of the GST sequences. Such fusion proteins can facilitate the purification of recombinant hG-CSF analog.

In another embodiment, the fusion protein is an hG-CSF analog containing a heterologous signal sequence at its N-terminus. In certain host cells (e.g., mammalian host cells), expression and/or secretion of hG-CSF analogs can be increased through use of a heterologous signal sequence. Such signal sequences are well known in the art.

Preferably, an hG-CSF analog fusion protein of the invention is produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). An hG-CSF analog-encoding polynucleotide can be cloned into such an expression vector such that the fusion moiety is linked in-frame to the hG-CSF analog.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The hG-CSF analog of the present invention also includes polypeptide conjugates with hG-CSF activity. The conjugates comprise a polypeptide moiety and at least one non-polypeptide moiety. In one embodiment, the non-polypeptide moiety is a 2-6 polyethylene glycol moiety. Compared to non-conjugated hG-CSF analog, the conjugates may have lower in vitro bioactivity, longer in vivo half-life, reduced receptor-mediated clearance and/or the ability to provide a more rapid stimulation of production of white blood cells and neutrophils.

Another aspect of the present invention relates to isolated polynucleotides encoding the hG-CSF of the present invention. The polynucleotide molecule of the present invention (i.e., the polynucleotide encoding the hG-CSF analog of the present invention and the polynucleotide molecule which is complementary to such a nucleotide sequence) can be generated using standard molecular biology techniques and the sequence information provided herein, as well as sequence information known in the art. For example, the polynucleotide encoding the hG-CSF analog may be generated by site-directed mutagenesis of a polynucleotide encoding the wild-type hG-CSF. Alternatively, the polynucleotide encoding the hG-CSF analog can be prepared by standard synthetic techniques, e.g., using an automated DNA synthesizer.

The polynucleotide molecule of the invention, moreover, can comprise only a portion of the polynucleotide sequence encoding the hG-CSF analog, for example, a fragment which can be used as a probe or primer. The probe/primer typically comprises a substantially purified oligonucleotide. The oligonucleotide typically comprises a region of nucleotide sequence that hybridizes under stringent conditions to at least about 7 or 15, preferably about 25, more preferably about 50, 75, 100, 125, 150, 175, 200, 225, 250, 275, 300, 325, 350, 400 or more consecutive nucleotides of the hG-CSF analog of the invention.

Probes based on the nucleotide sequence of the hG-CSF analog of the invention can be used to detect transcripts or genomic sequences corresponding to the hG-CSF analog of the invention. In preferred embodiments, the probe comprises a label group attached thereto, e.g., the label group can be a radioisotope, a fluorescent compound, an enzyme, or an enzyme co-factor. Such probes can be used as a part of a diagnostic kit for identifying cells or tissue which expresses the hG-CSF analog.

The invention encompasses all polynucleotide molecules that encode the same proteins due to degeneracy of the genetic code.

The invention also encompasses polynucleotide molecules which are structurally different from the molecules described above (i.e., which have a slight altered sequence), but which have substantially the same properties as the molecules above (e.g., encoded amino acid sequences, or which are changed only in non-essential amino acid residues).

In another embodiment, an isolated polynucleotide molecule of the invention is at least 15, 20, 25, 30, 50, 100, 150, 200, 250, 300, 350, 400, 450, 500, 550, 600, or more nucleotides in length and hybridizes under stringent conditions to a polynucleotide molecule corresponding to a nucleotide sequence of the hG-CSF analog of the invention. Preferably, the isolated polynucleotide molecule of the invention hybridizes under stringent conditions to the sequence of the hG-CSF analog.

The skilled artisan will further appreciate that changes can be introduced by mutation into the nucleotide sequences of the hG-CSF analog of the invention, thereby leading to changes in the amino acid sequence of the encoded proteins, without altering the functional activity of these proteins. An isolated polynucleotide molecule encoding the hG-CSF analog with a mutation can be created by introducing one or more nucleotide substitutions, additions or deletions into the nucleotide sequence of the polynucleotide molecule encoding the original hG-CSF analog, such that one or more amino acid substitutions, additions or deletions are introduced into the encoded protein. Such techniques are well known in the art. Mutations can be introduced into the hG-CSF analog of the invention by standard techniques, such as site-directed mutagenesis and PCR-mediated mutagenesis.

A polynucleotide may be further modified to increase stability in vivo. Possible modifications include, but are not limited to, the addition of flanking sequences at the 5′ and/or 3′ ends; the use of phosphorothioate or 2 O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Another aspect of the invention pertains to vectors containing a polynucleotide encoding the hG-CSF analog or a portion thereof. One type of vector is a “plasmid,” which includes a circular double-stranded DNA loop into which additional DNA segments can be ligated. In the present specification, “plasmid” and “vector” can be used interchangeably as the plasmid is the most commonly used form of vector. Vectors also include expression vectors and gene delivery vectors.

The expression vectors of the invention comprise a polynucleotide encoding the hG-CSF analog or a portion thereof in a form suitable for expression of the polynucleotide in a host cell, which means that the expression vectors include one or more regulatory sequences, selected on the basis of the host cells to be used for expression, and operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, such as the hG-CSF analog of the present invention.

The expression vectors of the invention can be designed for expression of the hG-CSF analog in prokaryotic or eukaryotic cells. For example, hG-CSF analog can be expressed in bacterial cells such as E. coli, insect cells (using baculovirus expression vectors), yeast cells such as S. cerevisiae or mammalian cells such as CHO cells. Alternatively, the expression vector can be transcribed and translated in vitro, for example using T7 promoter regulatory sequences and T7 polymerase.

The expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of the recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Examples of fusion expression vectors include pGEX (Pharmacia, Piscataway, N.J.), pMAL (New England Biolabs, Beverly, Mass.) and pRITS (Pharmacia, Piscataway, N.J.) which fuse glutathione S transferase (GST), maltose E binding protein, and protein A, respectively, to the target recombinant protein.

One strategy to maximize recombinant protein expression in E. coli is to express the protein in host bacteria with an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the hG-CSF analog expression vector is a yeast expression vector. Alternatively, the hG-CSF analog of the present invention can be expressed in insect cells using baculovirus expression vectors.

In yet another embodiment, a polynucleotide of the invention is expressed in mammalian cells using a mammalian expression vector. When used in mammalian cells, the expression vector's control functions are often provided by viral regulatory elements. For example, commonly used promoters are derived from polyoma, adenovirus 2, cytomegalovirus and Simian Virus 40.

The invention further provides gene delivery vehicles for delivery of polynucleotides to cells, tissues, or a mammal for expression. For example, a polynucleotide sequence of the invention can be administered either locally or systemically in a gene delivery vehicle. These constructs can utilize viral or non-viral vector approaches in in vivo or ex vivo modality. Expression of the coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle is preferably a viral vector and, more preferably, a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, togavirus viral vector.

The gene delivery vehicles of this invention are not limited to the abovementioned viral vectors. Other delivery methods and media may be employed such as, for example, nucleic acid expression vectors, polycationic condensed DNA linked or unlinked to killed adenovirus alone, ligand linked DNA, liposomes, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, DNA sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. Uptake efficiency of naked DNA may be improved using biodegradable latex beads. The method may be improved further by treatment of the beads to increase hydrophobicity and thereby facilitate disruption of the endosome and release of the DNA into the cytoplasm.

In addition, libraries of fragments of a protein coding sequence corresponding to the hG-CSF analog of the invention can be used to generate a diverse or heterogenous population of hG-CSF analog fragments for screening and subsequent selection of functional variants of an hG-CSF analog. In one embodiment, a library of coding sequence fragments can be generated by treating a double-stranded PCR fragment of an hG-CSF analog coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, an expression library can be derived which encodes N-terminal, C-terminal and internal fragments of various sizes of the hG-CSF analog.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify hG-CSF variants (Delgrave, et al. Protein Engineering 6:327-331, 1993).

Another aspect of the invention pertains to host cells into which a polynucleotide molecule of the invention is introduced. In one embodiment, the polynucleotide molecule contains sequences which allow it to homologously recombine into a specific site of the host cell's genome. In another embodiment, the polynucleotide molecule of the invention is introduced into the host cell by a viral or a non-viral vector. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the hG-CSF analog of the invention can be expressed in bacterial cells such as E. coli, insect cells, yeast or mammalian cells (such as Chinese hamster ovary cells (CHO), COS cells, Fischer 344 rat cells, HLA-B27 rat cells, HeLa cells, A549 cells, or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotides (e.g., DNA) into a host cell, including calcium phosphate or calcium chloride co-precipitation, DEAE-dextran-mediated transfection, lipofection, or electoporation.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotides encoding a selectable flag can be introduced into a host cell on the same vector as that encoding the hG-CSF analog of the invention or can be introduced on a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) the hG-CSF analog of the invention. Accordingly, the invention further provides methods for producing hG-CSF analog of the invention using the host cells of the invention. In one embodiment, the method comprises culturing the host cell of invention (into which a recombinant expression vector encoding the hG-CSF analog of the invention has been introduced) in a suitable medium such that hG-CSF analog of the invention is produced. In another embodiment, the method further comprises isolating hG-CSF analog of the invention from the medium or the host cell.

Another aspect of the present invention relates to a pharmaceutical composition comprising the hG-CSF analog and a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

In one embodiment, the active ingredients, which include the hG-CSF analog of the invention, are prepared with carriers that will protect the active ingredients against rapid elimination from the body, such as a controlled release formulation, implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form, as used herein, includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active ingredients and the particular therapeutic effect to be achieved, and the limitations inherent in the art of compounding such an active compound for the treatment of individuals.

In one embodiment, the hG-CSF analog of the invention is packaged in a dosage lower than the standard clinical dose of NEUPOGEN® (300 or 480 or 600 mcg per dose). In preferred embodiments, the hG-CSF analog of the invention is packaged in a dosage of between 1 and 200 mcg per day. In another embodiment, the hG-CSF analog of the invention is packaged in 50, 75 and 100 mcg doses.

Toxicity and therapeutic efficacy of the hG-CSF analog of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. The therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

In one embodiment, the hG-CSF analog is available as a preservative pharmaceutical composition comprising 50-500 mcg/ml of the hG-CSF analog. The composition can be administered subcutaneously without further admixture. Intravenous preparations require dilution with proper diluent, such as 5% dextrose, diluted to a final concentration of 1 to 25 mcg/ml. The pharmaceutical composition may contain human albumin to prevent adsorption to plastic materials during preparation and infusion. In one embodiment, the final concentration of human albumin is 2 mg/ml. The preservative pharmaceutical composition should be refrigerated at 2° C. to 8° C.

In another embodiment, the pharmaceutical composition of the present invention contains a small amount of acetate, Tween 80 and sodium.

The pharmaceutical compositions of the present invention may comprise the hG-CSF analog in a salt form. For example, because proteins can comprise acidic and/or basic termini side chains, the proteins can be included in the pharmaceutical compositions in either the form of free acids or bases, or in the form of pharmaceutically acceptable salts. Pharmaceutically acceptable salts can include suitable acids which are capable of forming salts with the proteins of the present invention including, for example, inorganic acids such as hydrochloric acid, hydrobromic acid, perchloric acid, nitric acid, thiocyanic acid, sulfuric acid, phosphoric acid, and the like; and organic acids such as formic acid, acetic acid, propionic acid, glycolic acid, lactic acid, pyruvic acid, oxalic acid, malonic acid, succinic acid, maleic acid, fumaric acid, cinnamic acid, anthranilic acid, citric acid, naphthalene sulfonic acid, sulfanilic acid and the like. Suitable bases capable of forming salts with the subject proteins can include, for example, inorganic bases such as sodium hydroxide, ammonium hydroxide, potassium hydroxide and the like; and organic bases such as mono-, di- and tri-alkyl amines (for example, triethyl amine, diisopropyl amine, methyl amine, dimethyl amine and the like) and optionally substituted ethanolamines (for example, ethanolamine, diethanolamine, and the like).

Although commercially available G-CSF is currently administered subcutaneously or intravenously, any method of administration that provides a therapeutically effective amount of the hG-CSF analog of the present invention can be used in the methods of the present invention. In one aspect, the hG-CSF analog can be in a variety of forms suitable for any route of administration, including, but not limited to, parenteral, enteral, topical or inhalation. Parenteral administration refers to any route of administration that is not through the alimentary canal, including, but not limited to, injectable administration, i.e., intravenous, intramuscular and the like as described below. Enteral administration refers to any route of administration which is oral, including, but not limited to, tablets, capsules, oral solutions, suspensions, sprays and the like, as described below. For purposes of this invention, enteral administration also refers to rectal and vaginal routes of administration. Topical administration refers to any route of administration through the skin, including, but not limited to, creams, ointments, gels and transdermal patches, as described below (see, also, Pharmaceutical Sciences, 18th Edition; Gennaro, et al., eds., Mack Printing Company, Easton, Pa., 1990).

Parenteral pharmaceutical compositions of the present invention can be administered by injection, for example, into a vein (intravenously), an artery (intraarterially), a muscle (intramuscularly) or under the skin (intradermally or subcutaneously) or in a depot composition.

The injectable pharmaceutical composition can be provided in powder form for reconstitution with a suitable vehicle, including but not limited to sterile pyrogen-free water, buffer, dextrose solution, etc., before use. To this end, the hG-CSF analog can be lyophilized as appropriate. The pharmaceutical compositions can be supplied in unit dosage forms and reconstituted prior to use in vivo.

Depot or sustained-release pharmaceutical compositions can be used in the methods of the invention. For example, continuous release of hG-CSF analog can be achieved by the conjugation of the hG-CSF analog with a water-soluble polymer as described in U.S. Pat. No. 5,320,840.

For prolonged delivery, the pharmaceutical composition can be provided as a depot preparation, for administration by implantation; e.g., subcutaneous, intradermal, or intramuscular. Thus, for example, the pharmaceutical composition can be formulated with suitable polymeric or hydrophobic materials (such as an emulsion in an acceptable oil) or ion exchange resins, or as sparingly soluble derivatives; as a sparingly soluble salt form of the hG-CSF analog, or derivative, mimetic or variant thereof. The hG-CSF analog can be present in an inert matrix or device for implantation to achieve prolonged release.

Alternatively, transdermal delivery systems manufactured as an adhesive disc or patch that slowly releases the active ingredient for percutaneous absorption can be used. To this end, permeation enhancers can be to facilitate penetration of the hG-CSF. A particular benefit may be achieved by incorporating the hG-CSF analog into a transdermal patch.

For oral administration, the pharmaceutical formulations can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art (see, Gennaro, et al., eds. Remington's Pharmaceutical Sciences, 18^{th} edition, Mack Printing Company, Pennsylvania, 1990).

Liquid pharmaceutical compositions for oral administration can take the form of, for example, solutions, syrups or suspensions, or they can be a dry product for constitution with water or other suitable vehicle before use. Such liquid pharmaceutical compositions can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid.).

The pharmaceutical compositions can also comprise buffer salts, flavoring, coloring and sweetening agents as appropriate. Pharmaceutical compositions for oral administration can be suitably prepared to provide controlled release of the hG-CSF analog.

Enteral pharmaceutical compositions can be suitable for buccal administration, for example, in the form of tablets, troches or lozenges. For rectal and vaginal routes of administration, the hG-CSF analog can be prepared as solutions (e.g., for retention enemas), suppositories or ointments. Enteral pharmaceutical compositions can be suitable for admixture in feeding mixtures, such as for mixture with total parenteral nutrition (TPN) mixtures or for delivery by a feeding tube (see, Dudrick, et al., 1998, Surg. Technol. Int. VII:174-184; Mohandas, et al., 2003, Natl. Med. J. India 16(1):29-33; Bueno, et al., 2003, Gastrointest. Endosc. 57(4):536-40; Shike, et al., 1996, Gastrointest. Endosc. 44(5):536-40).

For administration by inhalation, the hG-CSF analog can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated comprising a powder mix of the compound and a suitable powder base such as lactose or starch. Inhaled pharmaceutical compositions can be those, for example, described in U.S. Pat. Nos. 5,284,656 and 6,565,841, incorporated herein by reference in their entirety.

The compositions can, if desired, be presented in a pack or dispenser device that can comprise one or more unit dosage forms comprising the hG-CSF analog. The pack can, for example, comprise metal or plastic foil, such as a blister pack. The pack or dispenser device can be accompanied by instructions for administration.

The pharmaceutical compositions can be for a single, one-time use or can contain antimicrobial excipients, rendering the composition suitable for multiple, extended use with greater shelf stability, for example, a multi-use bottle. In another embodiment, the pharmaceutical composition of interest can be in unit dose or unit-of-use packages. As known in the art, a unit dose is targeted for a single use. The unit dose form can be in a vial, which can contain a solution or a desiccated form for reconstitution, a pre-filled syringe, a transdermal patch, and the like.

As is known to those of skill in the art, a unit-of-use package is a convenient prescription size, patient-ready unit labeled for distribution by health care providers. The package contains as much active ingredient as is necessary for a typical treatment regimen.

The pharmaceutical composition can be labeled and have accompanying labeling to identify the composition contained therein and other information useful to health care providers and end users. The information can include instructions for use, dose, dosing interval, duration, indication, side effects and other contraindications, warnings, precautions, storage recommendations and the like.

The invention provides methods of administering compositions of hG-CSF analog useful for preventing spontaneous abortion and implantation failure during assisted reproduction. The composition of hG-CSF analog can be administered by any route or on any schedule which provides a therapeutically or prophylactically effective amount of the hG-CSF analog.

In one embodiment, the composition of hG-CSF analog is administered parenterally. In a preferred embodiment, the composition of hG-CSF analog is administered subcutaneously or intravenously. The parenteral administration can be in a single bolus or as a continuous infusion. In one embodiment, the parenteral administration is a single intravenous infusion given over 15-30 minutes. In another embodiment, the parenteral administration is a continuous infusion of hG-CSF analog diluted in 5% dextrose.

### Kits

The invention also encompasses kits comprising the pharmaceutical composition of the present invention. These kits comprise one or more effective doses of the hG-CSF analog along with a label or labeling with instructions on using the hG-CSF analog according to the methods of the invention. These kits can also comprise components useful for carrying out the methods such as devices for delivering the hG-CSF analog and components for the safe disposal of these devices. Components of the kit may include, but are not limited to, diluents for reconstitution of unit dosages, syringes, needles, alcohol swabs, bandages, sharps bins, and instruction materials. The kit may further comprise hormone stimulating drugs in preparation for an IVF cycle. Typically, a kit may contain 5-60 doses of active ingredients. In one embodiment, the kit contains 30 doses of active ingredients.

### Computer Readable Media

Computer readable media comprising information of the hG-CSF analog of the invention is also provided. As used herein, “computer readable media” includes a medium that can be read and accessed directly by a computer. Such media include, but are not limited to, magnetic storage media, such as floppy discs, hard disc storage media, and magnetic tape; optical storage media such as CD-ROM; electrical storage media such as RAM and ROM; and hybrids of these categories such as magnetic/optical storage media. The skilled artisan will readily appreciate how any of the presently known computer-readable media can be used to create a manufacture comprising computer-readable medium having recorded thereon information of the hG-CSF analog of the invention.

As used herein, “recorded” includes a process for storing information on computer readable media. A variety of data processor programs and formats can be used to store the information of the present invention on computer-readable media. For example, the polynucleotide sequence corresponding to hG-CSF analog of the invention can be represented in a word processing text file, formatted in commercially-available software such as Microsoft Word and WordPerfect, or represented in the form of an ASCII file, stored in a database application, such as DB2, Sybase, Oracle, or the like. Any number of data processor structuring formats (e.g., text file or database) may be adapted in order to obtain computer readable medium having recorded thereon the hG-CSF analog of the present invention.

Another aspect of the present invention is directed to methods of preventing spontaneous abortion by administering to a subject in need thereof a prophylactically effective amount of the hG-CSF analog of the present invention.

The subject can be any mammalian subject at risk for a spontaneous abortion. In particularly preferred embodiments, the subject is a human female. In certain embodiments, the subject has previously had one or more spontaneous abortions. In further embodiments, the subject has previously had two or more spontaneous abortions. In other embodiments, the subject has had recurrent spontaneous abortions, i.e., three or more spontaneous abortions.

In further embodiments, the subject can be any subject in a population at risk for spontaneous abortion. For instance, the subject can be a human female in an age group at risk for spontaneous abortion. In particular embodiments, the subject can be a human female greater than 35 years of age, greater than 40 years of age or greater than 45 years of age. In other particular embodiments, the subject can be a human female less than 20 years of age or less than 15 years of age. However, essentially a woman of any age that presents with a reproductive infirmity, such as spontaneous abortion, preeclampsia and preterm labor, is a candidate for obtaining the materials and methods of the instant invention.

In further embodiments, the subject can also be in any other population at risk for spontaneous abortion as determined by a practitioner of skill in the art. In certain embodiments, the subject is threatening abortion. In other embodiments, the subject is obese, morbidly obese, has overall poor health or comorbid conditions that indicate a risk of spontaneous abortion to the skilled practitioner. In certain embodiments, these conditions can be incompetent cervix, uterine anomalies, hypothyroidism, diabetes mellitus, chronic nephritis, acute infection, use of illicit drugs (such as cocaine or crack), immunologic problems, severe emotional shock and viral infection (especially cytomegalovirus, herpes virus and rubella) (see, Merck Manual 17^{th} edition, 1999, Merck Research Laboratories, Whitehouse Station, N.J., p. 2053). In certain embodiments, the subject has had an implantation failure during a previous assisted reproduction procedure. Other subjects at risk include those with unusually high Th1 immune responses or unusually low Th₂ immune responses. In further embodiments, the subject can also be in any other population at risk for spontaneous abortion as determined by a practitioner of skill in the art.

In certain embodiments, the hG-CSF analog is administered to the subject prior to pregnancy. In one embodiment, the hG-CSF analog is administered to a subject that is planning or attempting to become pregnant. In other embodiments, the hG-CSF analog is administered to a pregnant subject. The hG-CSF analog can be administered at any time during the first or second trimester of pregnancy. In preferred embodiments, the hG-CSF analog is administered before and during the first 20 weeks of pregnancy.

The hG-CSF analog is administered in a prophylactically effective amount, i.e., an amount effective to reduce or eliminate the risk of spontaneous abortion in the subject. The amount can be determined by the skilled practitioner guided by the description herein and the knowledge in the art. In preferred embodiments, the amount can be any amount of hG-CSF analog that significantly inhibits apoptosis of trophoblast cells. Assays to determine apoptosis of trophoblast cells are well known to those of skill in the art (see, e.g., Sun Q H, et al., J. Interferon Cytokine Res. 2007 July; 27(7):567-78; Lecoeur H, et al., J. Immunol. Methods. 1997 Dec. 1; 209(2):111-23). In particular embodiments, a dose of 1 to 100 mcg/kg, 1 to 20 mcg/kg or about 10 mcg/kg is administered to the subject. In another embodiment, at least 25 mcg, at least 50 mcg, at least 75 mcg, at least 100 mcg, at least 125 mcg, at least 150 mcg, at least 175 mcg, at least 200 mcg, at least 300 mcg or more is administered daily.

The dose can be administered to the subject daily until the risk of spontaneous abortion is reduced or eliminated and as long as no symptoms of toxicity are presented. In certain embodiments, the dose is administered daily through the second trimester of pregnancy. In further embodiments, the dose is administered daily through the 20th week of pregnancy. In a particular embodiment, the dose is administered daily for four, three, two weeks or one week during the first or second trimester of pregnancy. In particular embodiments, the dose is administered daily for five to seven consecutive days before pregnancy. In particular embodiments, the dose is administered for five consecutive days during the first or second trimester of pregnancy. For example, the five consecutive days can be in the first or second week of pregnancy.

The hG-CSF analog can be administered according to any method of administration known to those of skill in the art. Preferred methods of administration include subcutaneous administration. Other effective modes of administration are described in detail in the sections below.

In certain embodiments, the hG-CSF analog is administered as a monotherapy. In other embodiments, the hG-CSF analog is administered with at least one other active compound. The hG-CSF analog and at least one other active compound can be administered simultaneously or sequentially, continuously or intermittently. For example, the other active ingredient can be administered according to the doses and schedules known to those of skill in the art while the hG-CSF analog is administered according to the methods described herein. The at least one other active compound can be another CSF. The other active compound can be a drug currently used to treat the conditions of interest. The other active compound can be a drug that is an immunosuppressant.

In preferred embodiments, the at least one other active ingredient is a chemotherapeutic or non-myeloablative immunosuppressive agent. For example, the other active ingredient can be cyclophosphamide or a purine nucleoside analog such as cladribine and fludararbine. Preferred chemotherapeutic or nommyeloablative immunosuppressive agents are described in detail in the sections below. The other active agent could also be another known immunosuppressive/anti-inflammatory agent such as vitamin D (or one of its analogs) or aspirin. In addition, the at least one other active agent could be one that is currently widely used for the treatment of Th1 cytokine excess in pregnancy, such as heparin, IVIG or progesterone.

In another aspect, the present invention provides methods of preventing embryo implantation failure during assisted reproduction by administration to a subject in need thereof a prophylactically effective amount of the hG-CSF analog of the present invention.

In vitro fertilization is an assisted procedure to overcome fertility problems caused by, for example, tubal disease, endometriosis, oligospermia, sperm antibodies and unexplained infertility. The procedure can include ovarian hyperstimulation with “fertility drugs” such as ovarian stimulants like clomiphene citrate and gonadotropin-releasing hormones. Hyperstimulation of the ovaries can induce growth of the egg (oocyte) and its encasing cells, collectively also termed the “ovarian follicles.” After sufficient follicular growth, final follicular maturation is induced and oocytes are retrieved or harvested. The oocytes are fertilized in vitro with sperm and the embryos cultured. A small number of embryos, generally 2-4, are then transferred to the uterus. Despite the transfer of multiple embryos, the term pregnancy rate is only about 25% (see, Merck Manual 17th edition, 1999, Merck Research Laboratories, Whitehouse Station, N.J., p. 1995).

In the methods of prevention, the hG-CSF analog is typically administered until implantation of the embryo to the uterine wall is achieved, until the risk of failed implantation is reduced or eliminated or according to the judgment of a practitioner of skill in the art.

In certain embodiments, the administration is continued until pregnancy is confirmed. In certain embodiments, the administration is started about the time of ovarian hyperstimulation and continued until about 3 days, about 5 days, about 7 days, about 10 days, about 12 days, about 14 days or about 30 days after embryo transfer to the subject's uterus. In certain embodiments, the administration is started about the time of ovarian hyperstimulation and continued until about the end of the first trimester. In another embodiment, the dose is administered for five to seven consecutive days prior to or about the time of embryo transfer to the subject's uterus.

In certain embodiments, a prophylactically effective amount of the hG-CSF analog is administered to a subject at risk of embryo implantation failure. In certain embodiments, a subject at risk is a subject that has failed one or more in vitro fertilization procedures. In further embodiments, the subject can also be in any other population at risk for failed embryo implantation as determined by a practitioner of skill in the art. In certain embodiments, the subject has previously failed assisted reproduction. In another embodiment, the subject has had one or more previous spontaneous abortions. In further embodiments, the subject can also be in any other population at risk for failed embryo implantation as determined by a practitioner of skill in the art.

In certain embodiments, the hG-CSF analog is administered to the subject prior to embryo transfer. For instance, the hG-CSF analog is administered to a subject that is planning or attempting to become pregnant via assisted reproduction. Thus the hG-CSF analog can be administered to the mother-to-be during the superovulation procedure or, if ova are donated, prior to implantation of the embryos. In other embodiments, the hG-CSF analog is administered to a subject after retrieving or harvesting oocytes. In another embodiment, the retrieved oocytes and the embryos are maintained and cultured in medium containing the hG-CSF analog prior to being instilled in the mother-to-be. The hG-CSF analog can be administered at any time during the assisted reproduction or in vitro fertilization process.

The methods provide for administration of the hG-CSF analog for a therapeutically or prophylactically effective time. In certain embodiments, the hG-CSF analog is administered prior to the onset or observation of the disorder or symptoms accompanying the disorder. In further embodiments, the hG-CSF analog is administered during the disorder or during the time period that symptoms accompanying the disorder are observed. In other embodiments, the hG-CSF analog is administered for a time after the disorder had cleared. For example, the hG-CSF analog can be administered about one day, about two days, about three days, about four days, about one week, about two weeks and up to about eight weeks, following resolution of the preeclampsia, signs of preterm labor, threatened abortion, or after confirmation of pregnancy during assisted reproduction.

The present invention is further illustrated by the following examples which should not be construed as limiting. The contents of all references, patents and published patent applications cited throughout this application, as well as the Figures and Tables, are incorporated herein by reference.

### Example 1

### Construction of Expression Vectors Containing the Coding Sequence of the hG-CSF Analogs

Plasmid containing coding sequence for wild type hG-CSF (SEQ ID NO:1) will be obtained from Codon Devices. Coding sequence for hG-CSF having substitutions at position 17 and at position 12, 16, 18, 22, 23, 32, 33, 38, 39, 43-46, 52, 53, 57, 58, 71, 77, 80, 83, 90, 93, 98, 101, 104, 105, 108, 115, 118, 122, 123, 137, 145, 159 or 169 will be created by site-directed mutagenesis.

The method for site directed mutagenesis and purification of the analogs will closely follow Reidhaar-Olson, et al. Mutations will be introduced using either cassette mutagenesis (Reidhaar-Olson et al., 1991; Wells et al., 1985) or primer-directed mutagenesis followed by restriction selection (Deng & Nickoloff, 1992; Wells et al., 1986). In the latter technique, oligonucleotide primers will be designed to introduce mutations at the desired codon and a silent change in a nearby restriction site. Restriction selection will be imposed before and after transformation into E. coli strain BMH 71-18 mutS (Zell & Fritz, 1987). Mutagenized plasmid DNA produced by either technique will be introduced into strain DH10B (Gibco BRL) by transformation, with selection for resistance to ampicillin. The bacteria will be grown and the plasmid will be isolated from the bacterial host and submitted for DNA sequencing to ensure the sequence of G-CSF nucleotides is correct with no mutations. The bacterial host carrying the plasmid will be grown, induced for protein expression, and tested by SDS-PAGE and Western blot to ensure the target protein is produced. In a small scale pilot study the bacterial host will be grown, induced, and target protein will be purified and assayed by the appropriate testing methods to determine yield, purity, and activity prior to scaling-up (Sanger et al., 1977).

The E. coli bacterial cells carrying the G-CSF-encoding plasmid (pG-CSF) grown under the conditions stated above will be harvested by centrifugation at 3500 g for 10 minutes. Cell pellets (example: approximately 2 g from 2 liters of culture) are resuspended in 1 mM DTT (approximately 10 ml) and passaged four times through a cell homogenizer at approximately 7000 PSI. The cell suspension is centrifuged at 10,000 g for 30 minutes, and the pellet is resuspended in 1% deoxycholate (DOC), 5 mM EDTA, 5 mM DTT, and 50 mM Tris, pH 9 (approximately 3 ml). The suspension is mixed at room temperature for 30 minutes followed by centrifugation at 10,000 g for 30 minutes. The pellet is resuspended in sterile water (approximately 4 ml) and centrifuged at 10,000 g for 30 minutes. The pellet is solubilized in 2% Sarkosyl and 50 mM Tris at pH 8 (approximately 1 ml). CuSO4 is added to 20 uM, the mixture is stirred 16 hours at room temperature, then centrifuged at 20,000 g for 30 minutes. The supernatant is harvested, and acetone is added (approximately 3 ml). The mixture is placed on ice for 20 minutes, then centrifuged at 5000 g for 30 minutes. The pellet is dissolved in 250 ml of 6M guanidine and 40 mM sodium acetate at pH 4, and the solution is loaded onto a G-25 column equilibrated in 20 mM sodium acetate, pH 5.4. The column is eluted with 20 mM sodium acetate at pH 5.4, and the peak is collected and loaded onto a CM-cellulose column equilibrated in 20 mM sodium acetate, pH 5.4. The column is washed with 20 mM sodium acetate at pH 5.4 and with 25 mM sodium chloride, followed by elution with 20 mM sodium acetate at pH 5.4 and 37 mM sodium chloride. The eluant is loaded onto a G-75 column equilibrated and run in 20 mM sodium acetate plus 100 mM sodium chloride at pH 5.4. The peak fraction is filter sterilized and endotoxins are removed by a commercial endotoxin removal kit (example: MiraCLEAN MIR 5900). The final concentration of G-CSF protein is determined (by A260/280 ratio and standard colorimetric protein assay) and yield is calculated by gel analysis (densitometric scanning of serial dilutions). Endotoxin/pyrogen level is determined commercially by the Limulus Amebocyte Lysate (LAL) test (Cambrex Corp., MD). Assays and tests of the physical and biological properties of the purified G-CSF protein are described elsewhere in this application.

Alternatively, expression vectors containing the coding sequence for hG-CSF analogs can be created using method described in U.S. Pat. No. 6,646,110. Briefly, the following DNA fragments will be synthesized following the general procedure described by Stemmer, et al., Gene 164:49-53 (1995).

Fragment 1, consisting of a Bam HI digestion site, a sequence encoding the YAP3 signal peptide, a sequence encoding the TA57 leader sequence, a sequence encoding a KEX2 protease recognition site (AAAAGA), a sequence encoding hG-CSF with substitutions at position 17 and at position 12, 16, 18, 22, 23, 32, 33, 38, 39, 43-46, 52, 53, 57, 58, 71, 77, 80, 83, 90, 93, 98, 101, 104, 105, 108, 115, 118, 122, 123, 137, 145, 159 or 169, as well as codon usage optimized for expression in E. coli and a Xba I digestion site.

Fragment 2, consisting of a Bam HI digestion site, a sequence encoding the YAP3 signal peptide, a sequence encoding the TA57 leader sequence, a sequence encoding a histidine tag, a sequence encoding a KEX2 protease recognition site, a sequence encoding hG-CSF with substitutions at position 17 and at position 12, 16, 18, 22, 23, 32, 33, 38, 39, 43-46, 52, 53, 57, 58, 71, 77, 80, 83, 90, 93, 98, 101, 104, 105, 108, 115, 118, 122, 123, 137, 145, 159 or 169, as well as codon usage optimised for expression in E. coli and a Xba I digestion site.

Fragment 3, consisting of a Nde I digestion site, a sequence encoding the OmpA signal peptide, a sequence encoding hG-CSF analog with its codon usage optimised for expression in E. coli and a Bam HI digestion site.

Fragment 4, consisting of a Bam HI digestion site, the Kozak consensus sequence (Kozak, M., J Mol. Biol. 1987 August 20; 196(4):947-50), a sequence encoding the hG-CSF signal peptide and a sequence encoding hG-CSF with substitutions at position 17 and at position 12, 16, 18, 22, 23, 32, 33, 38, 39, 43-46, 52, 53, 57, 58, 71, 77, 80, 83, 90, 93, 98, 101, 104, 105, 108, 115, 118, 122, 123, 137, 145, 159 or 169, as well as codon usage optimised for expression in CHO cells and a Xba I digestion site.

DNA fragments 1 and 2 were inserted into the Bam HI and Xba I digestion sites in plasmid pJSO37 (Okkels, Ann., New York Acad. Sci. 782:202-207, 1996) using standard DNA techniques. This resulted in plasmids pG-CSFcerevisiae and pHISG-CSFcerevisiae.

DNA fragment 3 was inserted into the Nde I and Bam HI digestion sites in plasmid pET12a (Invitrogen) using standard DNA techniques. This resulted in plasmid pG-CSFcoli.

DNA fragment 4 was inserted into the Bam HI and Xba I digestion sites in plasmid pcDNA3.1(+) (Invitrogen) using standard DNA techniques. This resulted in plasmid pG-CSFCHO.

### Example 2

### Expression of hG-CSF Analogs

HG-CSF analogs of the present invention will be expressed in mammalian and non-mammalian cells using the expression vectors produced in EXAMPLE 1.

(A) Expression of hG-CSF Analog in S. cerevisiae and E. coli.

Transformation of Saccharomyces cerevisiae YNG3 18 (available from the American Type Culture Collection, VA, USA as ATCC 208973) with either plasmid pG-CSFcerevisiae or pHISG-CSFcerevisiae, isolation of transformants containing either of the two plasmids, and subsequent extracellular expression of hG-CSF without and with the HIS tag, respectively, will be performed using standard techniques described in the literature. Transformation of E. coli BL21 (DE3) (Novagen, Cat. No. 69387-3) with pG-CSFcoli, isolation of transformants containing the plasmid and subsequent expression of hG-CSF in the supernatant and in the periplasm of the cell will be performed as described in the pET System Manual (8th edition) from Novagen.

Expression of the hG-CSF analog by S. cerevisiae and E. coli will be verified by Western Blot analysis using the ImmunoPure Ultra-Sensitive ABC Rabbit IgG Staining kit (Pierce) and a polyclonal antibody against hG-CSF (Pepro Tech EC Ltd.).

The expression levels of hG-CSF analog with and without the N-terminal histidine tag in S. cerevisiae and E. coli will be quantified using a commercially available G-CSF specific ELISA kit (Quantikine Human G-CSF Immunoassay, R&D Systems Cat. No. DCS50).

### Example 3

### Purification of hG-CSF Analog from S. cerevisiae Culture Supernatants

Cells will be removed by centrifugation. Cell depleted supernatant will be then filter sterilised through a 0.22 um filter. Filter sterilised supernatant will be diluted 5-fold in 10 mM sodium acetate pH 4.5. pH will be adjusted by addition of 10 ml concentrated acetic acid per 5 liters of diluted supernatant. The ionic strength should be below 8 mS/cm before application to the cation exchange column.

Diluted supernatant will be loaded at a linear flow rate of 90 cm/h onto a SP-sepharose FF (Pharmacia) column equilibrated with 50 mM sodium acetate, pH 4.5 until the effluent from the column reaches a stable UV and conductivity baseline. To remove any unbound material, the column will be washed using the equilibration buffer until the effluent from the column reaches a stable level with respect to UV absorbance and conductivity. The bound hG-CSF protein will be eluted from the column using a linear gradient; 30 column volumes; 0-80% buffer B (50 mM NaAc, pH 4.5, 750 mM NaCl) at a flow rate of 45 cm/h. Based on SDS-polyacryl amide gel electrophoresis, fractions containing hG-CSF analog will be pooled. Sodium chloride will be added until the ionic strength of the solution is more than 80 mS/cm.

The protein solution will be applied onto a Phenyl Toyo Pearl 650S column equilibrated with 50 mM NaAc, pH 4.5, 750 mM NaCl. Any unbound material will be washed off the column using the equilibration buffer. Elution of hG-CSF analog will be performed by applying a step gradient of MilliQ water. Fractions containing hG-CSF analog will be pooled. The purified protein will be quantified using spectrophotometric measurements at 280 nm and/or by amino acid analysis.

Fractions containing the hG-CSF analog will be pooled. Buffer exchange and concentration will be performed using VivaSpin concentrators (mwco: 5 kDa). The purified, concentrated hG-CSF analog may be further analyzed by SDS-PAGE. Amino acid analysis may also be performed on purified hG-CSF analog to confirm that the hG-CSF analog contain the expected amino acid residues based on the DNA sequence.

### Example 4

### Effect of the hG-CSF Analog on Trophoblast Cell Apoptosis

Purified hG-CSF analog will be tested for its ability to prevent apoptosis on JEG-3 cells exposed to recombinant human gamma interferon in in vitro culture.

The test will be performed using the method of Sun, et al. (Sun Q H, et al., J Interferon Cytokine Res. 2007 July; 27(7):567-78). Briefly, coriocarinoma cells (JEG or JAR-3 cell lines) will be exposed to recombinant human gamma interferon in vitro at a concentration that has been shown to induce apoptosis of cytotrophoblast cells (100 IU per ml) for 72 hours. The JEG or JAR-3 cells will be maintained in a chemically defined serum free culture media and will be grown in Teflon 24-well plates to prevent them from adhering. After 72 hours, the cell suspensions will be harvested and washed three times in PBS. Cells will then be stained with Annexin V and 7-AAD for analysis of cell death by flow cytometry (Lecoeur H, et al., J Immunol Methods. 1997 Dec. 1; 209(2):111-23). Cells that are Annexin V positive and 7-AAD negative will be scored as apoptotic. Cells that are negative for both Annexin V and 7-AAD will be scored as viable. Cells that are positive for both Annexin V and 7-AAD will be scored as nectrotic. The relative activity (the ratio of viable to apoptotic cells) of the analogs at various concentrations will be compared to that of gamma interferon alone and to a pseudowildtype hG-CSF analog. The pseudowildtype hG-CSF analog will contain a single substitution of an alanine for the native cysteine at position 17.

### Example 5

### The hG-CSF Analog of the Present Invention Prevents Embryotoxic Effects of Cells from Women with Recurrent Spontaneous Abortion In Vitro

In the in vitro clinical assay, mononuclear leukocytes will be isolated from women suffering from recurrent spontaneous abortion. The leukocytes will be cultured, and the culture medium will be removed from the leukocytes. This culture medium will be then contacted with murine embryos. Toxic factors in the culture medium typically kill the murine embryos in this assay.

The mononuclear leukocytes will be incubated with the hG-CSF analog prior to removal of the culture medium. The culture medium will be removed from the leukocytes and contacted with murine embryos. Survival of the murine embryos indicates reduction of embryotoxic factors in the culture medium and thereby the effectiveness of hG-CSF analog administration for prevention of spontaneous abortion in this in vitro model.

### Example 6

### The hG-CSF Analog of the Present Invention Prevents Spontaneous Abortion in a Mouse Model In Vitro

The murine mating pair CBA×DBA/2 (see, e.g., Yabuki, et al., 2003, Exp. Anim. 52(2)159-63) results in a spontaneous abortion rate of approximately 40%. In this example, female CBA mice will be treated according to the methods of the invention. Mice will be treated with hG-CSF analog prior to mating, at the time of mating and immediately after mating. A reduction of the rate of spontaneous abortion in mice treated with hG-CSF analog relative to control mice indicates that hG-CSF analog effectively prevents spontaneous abortion in this in vivo model.

### Example 7

### Patients' Case Studies

Over the course of the last 4 years, three patients undergoing assisted reproduction procedures have been treated with recombinant hG-CSF (rhG-CSF). Case studies of these three patients are provided below.

(1) JC

J.C. is a 36-year-old married white female with an obstetrical history of three uncomplicated vaginal deliveries at full term (all male children) followed by six consecutive first trimester miscarriages (each at 10-12 weeks). Conception was natural in each of the successful pregnancies and in each miscarriage. Each miscarried fetus was karyotyped, and all were normal. The couple then experienced three years of secondary infertility. At that point, she sought a consultation with a reproductive endocrinologist (RE).

The RE performed a detailed workup to attempt to identify the cause of the couple's reproductive failures. No anatomic or endocrinologic etiology was identified. Both J.C. and her husband were found to be karyotypically normal. A standard andrology workup for the husband was negative.

J.C.'s past medical history was significant in that J.C. had a remote past history of seasonal allergies and ten years of allergy desensitization shots. Based on this medical history, a series of immunologic tests including measurement of Th1 and Th2 cytokine production in vitro were ordered. As noted previously in this application, allergy is a classic Th2 immunopathologic response. Although few allergists realize it, allergy desensitization works by presenting the allergen in a manner that favors Th1 cytokine production instead of Th2 cytokine production. In many individuals, this shift from Th2 to Th1 dominance becomes more generalized and antigen non-specific. The series of tests ordered for J.C. specifically measured Th1/Th2 cytokines produced _{by} the patient's peripheral blood mononuclear cells (PBMC) in response to the non-specific mitogen phytohemagglutinin (PHA). J.C.'s PBMC produced greater than 10,000 units per ml of the prototypic Th1 cytokine gamma interferon in response to PHA. Levels of the prototypic Th2 cytokine IL-4 and the counter regulatory Th2 cytokine IL-10 were undetectable.

The RE performed intrauterine insemination (IUI) using J.C.'s husband's sperm. The first attempt at IUI resulted in a positive HCG at 7 days. The rhG-CSF administration was initiated the following day. The regimen consisted of 100 mcg/day of rhG-CSF (Neupogen) injected subcutaneously for a total of 30 days, a cumulative dose of 3000 mcg. The rhG-CSF regimen was carried out for the full 30 days and then discontinued. The patient experienced no rhG-CSF-related side effects at any point during the regimen.

At day 14 of the rhG-CSF regimen, another blood sample was obtained from J.C. for repeat measurement of Th1 and Th2 cytokines by her PBMC in response to PHA. The repeat results showed undetectable levels of the prototypic Th1 cytokine gamma interferon and elevated levels (2,000 units per ml) of the counter regulatory Th₂ cytokine IL-10. These results clearly indicated that rhG-CSF produced a shift from Th1 to Th2 cytokine production by her PBMC in response to PHA. Interestingly, J.C.'s allergies had also returned. This is consistent with the shift from Th1 to Th2 cytokine dominance.

At 8 weeks, an ultrasound confirmed an ongoing healthy pregnancy with a well-formed gestational sac and a fetus with a strong heartbeat. The pregnancy continued to progress uneventfully and at 11 weeks J.C. was transferred from the care of her RE to the care of a general obstetrician. The pregnancy progressed without complication, and a healthy 8 lb., 19-inch female was delivered by planned cesarean section at 38 weeks. Mother and child are both doing well.

(2) NC

N.C. is a healthy 35-year-old married white female with an obstetrical history of primary infertility including three failed IUIs and one failed IVF.

N.C.'s first IUI resulted in monozygotic twins, one of which revealed no fetal heartbeat at 6 weeks and the other which had a confirmed weak fetal heartbeat at 6 weeks but no heartbeat by the 7^{th} week. The second IUI resulted in a singleton pregnancy and fetal demise at 8 weeks. A heartbeat was seen at the 7^{th} week but was negative by the 8^{th} week. Karyotyping was performed and revealed an abnormal karyotype (69 XXY). N.C.'s third IUI resulted in a probable ectopic pregnancy treated with methotrexate. N.C.'s last pregnancy attempt was a cycle of IVF. This resulted in a confirmed and apparently healthy pregnancy at 6 weeks with a gestational sac measuring 36×37 millimeters and fetal heart rate of 113. However, one week later no fetal heartbeat was observed. The products of conception were expelled in large clots, and karyotyping was performed. Karyotyping was revealed to be normal (46 XY). N.C.'s RE performed an exhaustive workup to determine the cause of her reproductive failures. However, the workup failed to reveal any identifiable cause.

N.C.'s past medical history was non-contributory. She appeared to be a healthy female with unexplained primary infertility and repeated pregnancy loss. A review of her medical records revealed past laboratory testing showed a normal balance of Th1 and Th2 cytokines.

Because one of N.C.'s early losses involved a karyotypically abnormal embryo (69 XXY), N.C. had arranged for preimplantation genetic diagnosis for her last (failed) IVF cycle. N.C. had two cryopreserved embryos left from that cycle, and those embryos were used for the IVF cycle with rhG-CSF. N.C. received 100 mcg per day for the seven days prior to transfer and for 30 additional days after transfer, at a cumulative dose of 3700 mcg. N.C. experienced no rhG-CSF related side effects. At 6 weeks an ultrasound evaluation of N.C. revealed a healthy pregnancy with a well-formed gestational sac (40×40 mm) and a strong heart beat (145 beats per minute). At the 10th week, N.C. was transferred from her RE's care to the high-risk obstetrical unit in a hospital where she delivered a healthy baby boy. Both mother and child are doing well.

Approximately one year later, N.C. opted to undergo another IVF cycle at a different clinic without the benefit of rhG-CSF therapy. This cycle failed and was classified as a biochemical pregnancy (positive beta HCG, no evidence of gestational sac or embryo).

A few months later, N.C. contacted the inventor to request that he provide consultation regarding the use of rhG-CSF in her next IVF cycle. The inventor agreed and a clinical plan identical to her previous IVF cycle using rhG-CSF was pursued. N.C. began rhG-CSF (100 mcg per day) five days prior to embryo transfer (i.e., on the day of oocyte retrieval) in a fresh IVF cycle. The pregnancy is ongoing and her RE has transferred her to the care of a general obstetrician. At her last examination (at 20 weeks), all measurements were normal for gestational age and fetal heartbeat was strong.

(3) JJ

J.J. is a 33-year-old married white female with a history of primary subfertility and seven failed pregnancies. Over a period of three years, J.J. suffered three first-trimester miscarriages and three chemical pregnancies. Four of the pregnancies involved the use of fertility drugs and natural conception. Two of the pregnancies occurred through IUI. The last pregnancy was a failed cycle of IVF.

J.J.'s RE performed a standard workup to attempt to determine cause for J.J.'s failures. The workup failed to identify a cause. Both members of the couple were found to be karyotypically normal. J.J. and her RE decided that she should consult with a Reproductive Immunologist. Prior to J.J.'s IVF cycle, this physician performed a battery of laboratory tests and a medical evaluation and concluded that J.J. should undergo a course of Intravenous Immunoglobulin (IVIG) to correct immune problems identified through testing. Repeat laboratory tests demonstrated that IVIG failed to correct the purported immunologic problem. J.J.'s IVF cycle resulted in an ectopic pregnancy, and J.J. required emergency surgery for a unilateral salpingectomy.

J.J. and her RE sought a consultation with the inventor and decided to undergo another cycle of IVF with rhG-CSF treatment.

J.J. underwent another cycle of IVF with frozen embryos from her previous cycle. Although J.J. was scheduled to begin rhG-CSF at 100 mcg per day five days prior to embryo transfer, J.J. was not able to begin rhG-CSF until three days before embryo transfer. The rhG-CSF was continued at 100 mcg per day for 30 days after embryo transfer. The cumulative dose of rhG-CSF was 3300 mcg. J.J. completed her course of rhG-CSF and experienced no rhG-CSF related side effects.

Two embryos were transferred. The cycle resulted in a positive beta HCG (139 at 7 days post transfer; 316 at 10 days post transfer). Six weeks post transfer, an ultrasound identified a well-formed gestational sac and a heart beat of 115.

J.J. underwent another ultrasonic evaluation at 10 weeks gestation, and a strong heartbeat was identified and all measurements were exactly appropriate for dates. J.J. was transferred to the care of a general obstetrician and delivered a healthy baby girl. Both the mother and the child are healthy and doing well.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

is the amino acid sequence of the
wild-type human G-CSF.
SEQ ID NO: 1
TPLGPASSLP QSFLLKCLEQ VRKIQGDGAA LQEKLCATYK
LCHPEELVLL GHSLGIPWAP LSSCPSQALQ LAGCLSQLHS
GLFLYQGLLQ ALEGISPELG PTLDTLQLDV ADFATTIWQQ
MEELGMAPAL QPTQGAMPAF ASAFQRRAGG VLVASHLQSF
LEVSYRVLRH LAQP

1-20. (canceled)

21. A method for reducing the likelihood of spontaneous abortion in a subject in need thereof, comprising administering to the subject an effective amount of an hG-CSF analog which comprises an amino acid sequence that differs from the sequence of SEQ ID NO:1 at position 17 and at least one other position.

22. The method of claim 21, wherein the hG-CSF analog is administered before and during the first trimester of pregnancy.

23. The method of claim 21 wherein the hG-CSF analog is administered parenterally or subcutaneously.

24. (canceled)

25. The method of claim 21, wherein the hG-CSF is co-administered with an immunosuppressive or chemotherapeutic agent.

26. The method of claim 25, wherein the immunosuppressive agent is cyclophosphamide, cladibrine, or fludarabine.

27. A method for reducing the likelihood of implantation failure during assisted reproduction in a subject in a need thereof comprising administering to the subject an effective amount of the hG-CSF analog which comprises an amino acid sequence that differs from the sequence of SEQ ID NO:1 at position 17 and at least one other position.

28. The method of claim 27, wherein the subject is treated prior to transfer of an embryo into the subject.

29. The method of claim 27, wherein the subject is treated from the time the embryos are transferred into the subject.

30. The method of claim 29 wherein the hG-CSF analog is administered parenterally or subcutaneously.

31. The method according to claim 21, wherein said amino acid sequence differs from SEQ ID NO:1 in that the cysteine residue at amino acid position 17 of SEQ ID NO:1 is substituted with an amino acid selected from the group consisting of leucine, methionine, glutamine, tryptophan, alanine, tyrosine, serine, lysine, glutamine, threonine, asparagine, and histidine.

32. The method according to claim 21, wherein said amino acid sequence differs from SEQ ID NO: 1 at amino acid positions 17 and 38.

33. The method according to claim 21, wherein said amino acid sequence differs from SEQ ID NO: 1 at amino acid positions 17, 38 and 53.

34. The method according to claim 21, wherein said amino acid sequence differs from SEQ ID NO: 1 at amino acid positions 17, 38 and 58.

35. The method according to claim 21, wherein said amino acid sequence differs from SEQ ID NO: 1 at amino acid positions 17, 38, 53 and 58.

36. The method according to claim 21, wherein said amino acid sequence differs from SEQ ID NO: 1 at amino acid position 17 and at one or more positions selected from the group consisting of position 12, 16, 18, 23, 32, 33, 43-46, 52, 57, 58, 71, 83, 90, 98, 101, 104, 108, 123, 137 and 159.

37. The method according to claim 21, wherein said amino acid sequence differs from SEQ ID NO: 1 at amino acid position 17 and at one or more positions selected from the group consisting of position 22, 38, 39, 53, 77, 80, 93, 105, 115, 118, 122, 145 and 169.

38. The method according to claim 21, wherein said hG-CSF analog is administered at a dose about 1-100 mcg/kg.

39. The method according to claim 21, wherein said hG-CSF analog is administered at a dose about 1-20 mcg/kg.

40. The method according to claim 21, wherein said hG-CSF analog is administered at a dose about 1-10 mcg/kg.

41. The method according to claim 27, wherein said amino acid sequence differs from SEQ ID NO:1 in that the cysteine residue at amino acid position 17 of SEQ ID NO:1 is substituted with an amino acid selected from the group consisting of leucine, methionine, glutamine, tryptophan, alanine, tyrosine, serine, lysine, glutamine, threonine, asparagine, and histidine.

42. The method according to claim 27, wherein said amino acid sequence differs from SEQ ID NO: 1 at amino acid positions 17 and 38.

43. The method according to claim 27, wherein said amino acid sequence differs from SEQ ID NO: 1 at amino acid positions 17, 38 and 53.

44. The method according to claim 27, wherein said amino acid sequence differs from SEQ ID NO: 1 at amino acid positions 17, 38 and 58.

45. The method according to claim 27, wherein said amino acid sequence differs from SEQ ID NO: 1 at amino acid positions 17, 38, 53 and 58.

46. The method according to claim 27, wherein said amino acid sequence differs from SEQ ID NO: 1 at amino acid position 17 and at one or more positions selected from the group consisting of position 12, 16, 18, 23, 32, 33, 43-46, 52, 57, 58, 71, 83, 90, 98, 101, 104, 108, 123, 137 and 159.

47. The method according to claim 27, wherein said amino acid sequence differs from SEQ ID NO: 1 at amino acid position 17 and at one or more positions selected from the group consisting of position 22, 38, 39, 53, 77, 80, 93, 105, 115, 118, 122, 145 and 169.

48. The method according to claim 27, wherein said hG-CSF analog is administered at a dose about 1-100 mcg/kg.

49. The method according to claim 27, wherein said hG-CSF analog is administered at a dose about 1-20 mcg/kg.

50. The method according to claim 27, wherein said hG-CSF analog is administered at a dose about 1-10 mcg/kg.

51. The method of claim 27, wherein the hG-CSF is co-administered with an immunosuppressive or chemotherapeutic agent.
